(19) United States
(12) Patent Application Publication
(10) Pub. No.: US 2020/0405901 A1
(43) Pub. Date: Dec. 31, 2020

(54) PORTABLE OZONIZING DEVICE

(71) Applicant: Renzo SACCARDO, Vigodarzere (PD) (IT)

(72) Inventor: Renzo SACCARDO, Vigodarzere (PD) (IT)

(21) Appl. No.: 16/912,921

(22) Filed: Jun. 26, 2020

(30) Foreign Application Priority Data
Jun. 26, 2019 (IT) 102019000010179

(51) Int. Cl. A61L 9/12; B01D 53/00; B01D 53/34; C01B 13/11

(52) U.S. Cl. CPC A61L 9/122; B01D 53/007; B01D 53/346; C01B 13/11; F24F 2003/1685; C01B 2201/12; C01B 2201/82; A61L 2209/111; A61L 2209/134; B01D 2259/804

(57) ABSTRACT

An ozonizing device comprises a box-shaped body which encloses therein ventilation device, a discharge ozone generator, an ozone-catalysing UV-C lamp and an additional ozone generator. The box-shaped body comprises a partition wall intended to define two distinct chambers in said box-shaped body: a first chamber in which the ventilation device, the discharge ozone generator and the ozone-catalysing UV-C lamp are housed, and a second chamber in which the further ozone generator is housed which is interfaced with air distribution device associated with a diffuser tube for the diffusion of an ozone-enriched air flow. The ozonizing device further comprises a control unit configured so as to selectively activate, alone or in combination, each of the discharge ozone generator, ozone-catalysing UV-C lamp and the additional ozone generator.

### FIELD OF THE INVENTION

The present invention relates to an ozonizing device of the type comprising a box-shaped casing inside which at least one discharge ozone generator and a UV-C lamp for converting the ozone into oxygen are housed.

### BACKGROUND OF THE INVENTION

Within the technical field of reference, it is known to use ozone generators in order to carry out an air purification treatment.

For this purpose, portable devices are known which allow ozone to be introduced into a room or any closed environment by means of air recirculation.

Typically, these apparatuses comprise an outer casing inside which there are ozone production means and one or more fans which allow an air flow to be created which is suctioned in from the outside, impinges the aforementioned means and is re-introduced into the environment charged with ozone particles.

One of the main drawbacks associated with known apparatus relates to the fact that an excessive presence of ozone in an environment may be harmful to people.

For this purpose, patent application WO 2007/105099 suggests combining the ozone generator with a catalyst, specifically formed by a UV lamp with appropriate wavelength, in order to destroy the ozone before it is released into the environment.

Although this solution is effective in limiting the quantity of ozone emitted, it is evident that it entails the strong drawback of not allowing an appropriate sanitization of the environment since only the part of air close to the apparatus and which circulates therein is actually purified, while the rest of the room cannot be effectively reached by the action of the apparatus.

Furthermore, the immediate destruction of ozone following its generation and the consequent reduced quantity of ozone actually released into the environment necessarily entails an extension of the times for the purification activity.

It is therefore desirable to provide new solutions for the construction of ozonizing apparatuses which are able to avoid, or in any case contain, the problems deriving from the presence of ozone in closed environments.

There is also a need to have available ozonizing apparatuses which are able to operate in a more versatile manner, better adapting to the different operating needs that may arise.

Therefore, the problem underlying the present invention is that of providing an ozonizing device which is structurally and functionally conceived to overcome the drawbacks that have emerged with reference to the above prior art.

A further object is to provide an ozonizing device which makes it possible to eliminate the problems and risks associated with the residual ozone in the environments where sanitization has been carried out.

Another object of the present invention is to provide an ozonizing device which is particularly versatile and simple to use, allowing it to adapt appropriately to the different operating needs of the users, without inducing them to involuntary errors, which may even be potentially dangerous and/or serious.

### SUMMARY OF THE INVENTION

Such a problem is solved and such objects are achieved by the invention by means of an ozonizing device made according to claim 1.

It will be appreciated that the ozonizing device according to the present invention makes it possible to differentiate the ozone production according to the actual needs by virtue of the combined presence of an ozone generator, preferably a discharge ozone generator, a UV-C lamp and an additional ozone generator combined with air distribution device which may be selectively operated by means of the associated control unit.

In this way it is possible to use the most appropriate ozone production device according to the needs, in particular by limiting the production of ozone in the case of lightly soiled environments or where only a modest purification is required, and/or only deodorization.

Preferably, the discharge ozone generator and the ozone-catalysing UV-C lamp, for the destruction of the residual ozone, are housed in a first chamber defined in the device of the present invention, while the further ozone generator is housed in a second chamber thereof, allowing the overall dimensions to be optimized and the device to be made more efficient during operation.

According to another aspect of the invention, the user may select a series of operating sequences, or predefined programs, in such a way as to be able to operate the device according to the actual needs in a simple intuitive manner and in complete safety for the users.

Further preferred features of the invention are more generally defined by the dependent claims.

### BRIEF DESCRIPTION OF THE DRAWINGS

The features and the advantages of the invention will become more apparent from the detailed description of some exemplary embodiments thereof illustrated, by way of non-limiting example, with reference to the accompanying drawings, in which:

FIG. 1 is a front perspective view of the ozonizing device according to the present invention;

FIG. 2 is a rear perspective view of the ozonizing device according to the present invention;

FIG. 3 is a perspective view of the ozonizing device according to the present invention from which a lid element has been removed to illustrate some internal components of the device;

FIGS. 4A and 4B are two perspective views, according to opposite observation points, of a partition wall of the device of the present invention;

FIG. 5 is a perspective view of the lid element of the device of the present invention; and

FIG. 6 is a schematic drawing illustrating the components of the ozonizing device according to the present invention.

### DETAILED DESCRIPTION OF THE INVENTION

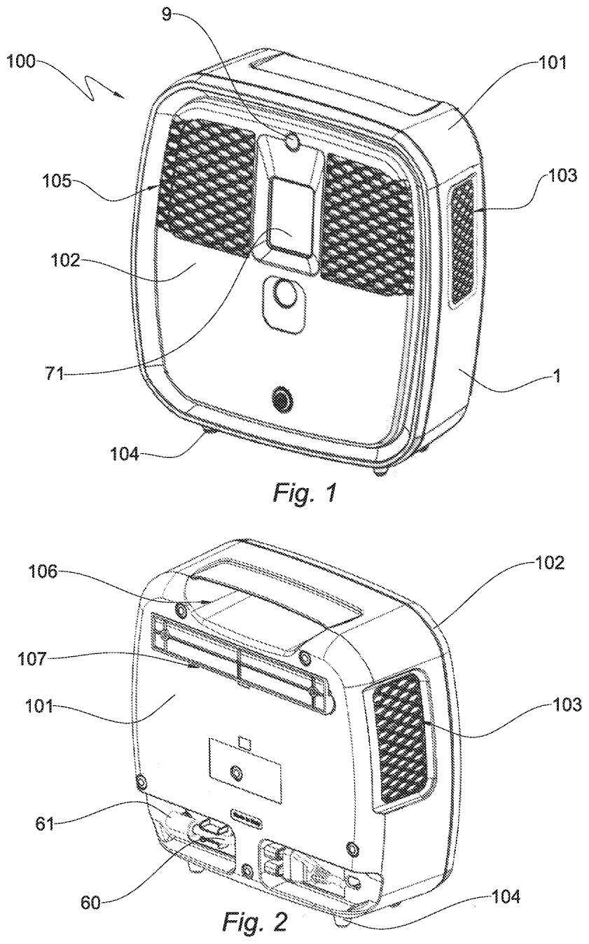

With reference initially to FIG. 1, an ozonizing device according to the present invention is indicated globally with the reference numeral 100. The ozonizing device 100 is preferably of the portable type, that is to say it has dimensions and shapes which make it easily transportable by a user and does not require a fixed installation, but connection to a common household electric power supply or other source of energy, including accumulators.

The ozonizing device 100 comprises a box-shaped body 1 which contains the components intended for the operation thereof, as will be illustrated in greater detail below.

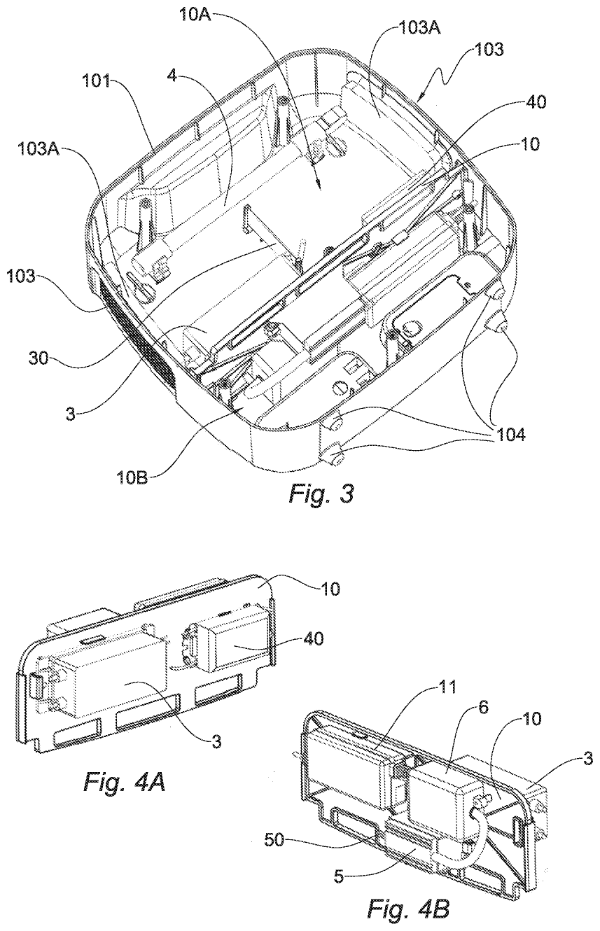

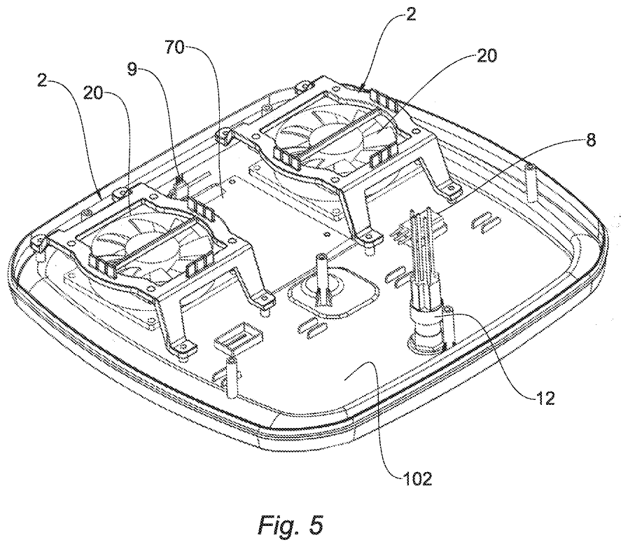

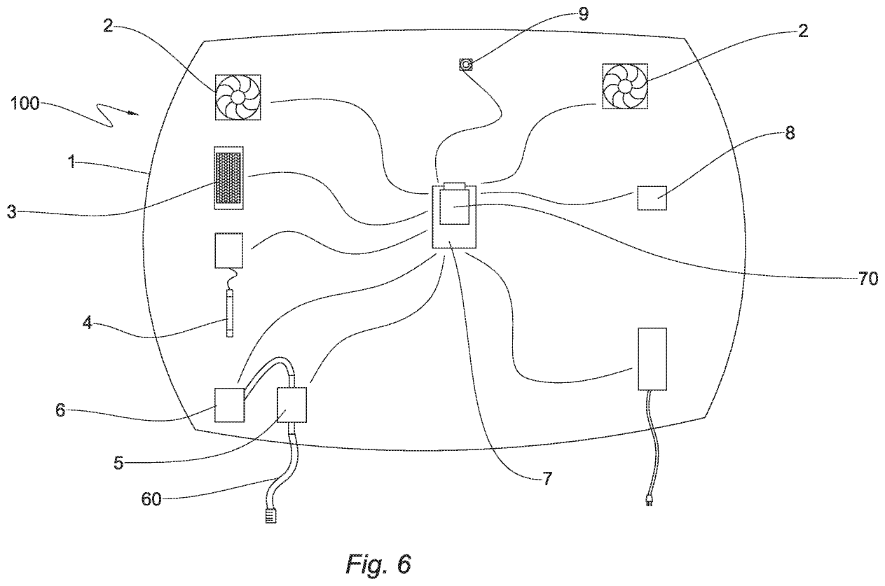

Preferably, the box-shaped body is formed by a main body 101, to which a lid element 102 is coupled, capable of closing box-like main body 101 and on which some of the components of the device 100 may be advantageously fixed. The main body 101 and the lid element 102 are better illustrated in FIGS. 3 and 5, respectively.

In some embodiments, the box-shaped body 1 is provided with a handle 106, to facilitate transport thereof, and, preferably, with one or more support feet 104, as shown in the exemplary embodiment shown in FIG. 2.

In addition or as an alternative to the handle 106 there may also be a quick coupling/release system 107, by means of which the device 100 may also be positioned on vertical walls.

For example, such a system may be implemented by means of a longitudinal bar which defines an undercut in which a hook fixed to the wall may be inserted. However, different solutions may also be contemplated which allow a removable attachment of the device to a wall.

Such a system advantageously allows rapid fixing in safety.

With reference now to FIG. 3, inside the box-shaped body 1 there are ventilation device 2, preferably formed by one or more fans 20. The ventilation device 2 are configured in such a way as to create an air flow which is taken from outside the device 100 and which, after passing through a first chamber 10A defined inside the box-shaped body 1, in a way that will be illustrated below, is brought out again.

For this purpose, lateral apertures 103 and/or front apertures 105 in the box-shaped body 1, illustrated in FIG. 1, may be defined for the intake/discharge of air into/from said first chamber 10A. Preferably, such apertures are arranged facing the first chamber 10A, so that the air flow mentioned above takes place within said chamber 10A. In some embodiments, the apertures 103 and 105 may be associated with respective optical and anti-insect filters 103A, for example of the terry-weave type.

Inside the box-shaped body 1, preferably at the first chamber 10A, there are also one or more discharge ozone generators 3 and one or more ozone-catalysing UV-C lamps 4, therefore capable of destroying ozone, converting it into molecular oxygen, as well as preferably also the ventilation device 2.

Such discharge ozone generator 3 and ozone-destroying UV-C lamp 4 are configured in such a way as to treat the air flow generated by the ventilation device 2, thus enriching the air in the environment with ozone, followed by the post-sanitization destruction.

Advantageously, the device according to the present invention further comprises a control unit 7 configured in such a way as to selectively activate, alone or in combination, the discharge ozone generator 3 and the ozone-destroying UV-C lamp 4.

As mentioned above, these devices may be activated according to the user's needs and, in general, the use of the UV-C lamp will be preferred for low-intensity applications, such as for example in order to freshen the air in an environment, removing the odours present, while the discharge generator will mainly be used if greater purification is required.

The selection may be made by the user via a control panel 71, preferably formed by a touch screen 70, through which the user may select a predefined operating sequence of the device 100.

However, it will be appreciated that as an alternative or in combination with the touch screen, the use of a separate push-button panel and/or a Wi-Fi and/or remote radio application, as well as a connection and control system via the Internet which allows control of the remote device, may be provided.

According to an aspect of the invention, the control unit 7 is in fact configured in such a way as to store a set of predefined and safe operating sequences, each representative of a device operating program. Such sequences may include the time duration of the ozone production and the possible alternation of operation of the various ozone generating devices present in the device, as well as other functions illustrated below. It will also be appreciated that, in some embodiments, the device 100 comprises one or more ionizing devices 8 which are also interfaced to the control unit 7 and which may also be alternatively activated or deactivated according to the program selected by the user. Preferably, the ionizer device 8 is also housed in the first chamber 10A.

According to another aspect, the ozonizing device 100 may also comprise one or more presence sensors 9 configured to detect the presence of persons and/or animals inside the room in which the device 100 is positioned.

The presence sensor 9 is advantageously interfaced with the control unit 7 in such a way as to interrupt the generation of ozone following the detection of persons and/or animals, either stationary or moving.

It will be appreciated that the presence sensor 9 may be active or inactive according to the specific programs selected by the user or in general according to the specific operating sequence. For example, in the case of air purification using only the UV-C lamp, in which there is no ozone production, the sensor is deactivated, thus allowing the operation of the ozonizing device 100 even in the presence of people. On the contrary, when the discharge generator is active, the sensor is activated, avoiding possible risks for people.

According to a further aspect, the discharge ozone generator 3 comprises one or more ozone generating plates 30.

It will therefore be appreciated that, in some embodiments, the ventilation device 2 may comprise two or more fans 20 and, in the illustrated embodiment in which a plate and two fans are present, it is possible to advantageously arrange the ozone generating plate 30 with this being arranged in an intermediate position between the two fans 20, so as to optimize the air flow which impinges it.

According to yet another aspect, at least one fan of the ventilation device 2 is located in the first chamber 10A in an intermediate position between the discharge ozone generator 3 and said UV-C lamp, 4, thus contributing to the rationalization of the dimensions.

With reference now to FIGS. 3 and 4A-B, the ozonizing device 100 preferably comprises a partition wall 10 which divides the box-shaped body 1 into two, defining the first chamber 10A and a second chamber 10B.

Preferably, the second chamber 10B is arranged, in use, below the first chamber 10A. It will, however, be appreciated that in alternative embodiments the second chamber 10B may be arranged above, side by side, placed in front of or behind the first chamber 10A.

An additional ozone generator 5 is advantageously housed in the second chamber 10B. Such a further ozone generator 5, preferably a discharge ozone generator but of smaller size and capacity than the ozone generator 3 housed in the first chamber 10A, is interfaced with air distribution device 6, for example a compressor, associated with a diffuser tube 60 for the diffusion of an ozone-enriched air flow outside the ozonizing device 100.

In preferred embodiments, the diffuser tube 60 is housed in and removable as needed from the box-shaped body 1 and may have a diffuser 61 placed at the free end thereof.

The diffuser tube may be advantageously immersed in a container with water or other liquid, allowing the purification thereof by means of ozone. The further ozone generator 6 is also interfaced with the control unit 7 and, similarly to that illustrated above for the other ozone generators, may be selectively activated according to the user's needs. It should also be noted that, preferably, the discharge ozone generator 3, the ozone-destroying UV-C lamp 4 and the further ozone generator 5 are mounted on the partition wall 10.

Also in this case, in alternative embodiments, mounting on vertical and back walls, as well as front or even upper covers may be provided.

Therefore, an ozonizing device according to the invention makes it possible to easily adapt to the different needs of the users, with a compact and rational solution.

The production and release of ozone may always be regulated according to the actual need, thus allowing the problems deriving from an excessive presence of ozone in a room to be limited.

1. An ozonizing device comprising a box-shaped body which encloses therein a ventilation device, a discharge ozone generator, an ozone-catalysing UV-C lamp for destroying ozone, and an additional ozone generator, the box-shaped body including a partition wall which is intended to define two different chambers in the box-shaped body, a first chamber in which the ventilation device, the discharge ozone generator and the ozone-catalysing UV-C lamp are received and a second chamber, in which the additional ozone generator is received, said additional ozone generator being interfaced with air distribution device which is associated with a diffuser tube for diffusing an air flow which is enriched with ozone outside said ozonizing device by said diffuser tube, said first chamber being configured in such a manner that an air flow through said box-shaped body is defined, the ozonizing device further comprising a control unit which is configured so as to selectively activate, alone or in combination, each of said discharge ozone generator, ozone-catalysing UV-C lamp and additional ozone generator.

2. The ozonizing device according to claim 1, further comprising an ionizing device which is interfaced with said control unit.

3. The ozonizing device according to claim 1, further comprising a presence sensor which is configured to detect the presence of persons and/or animals inside a room in which the ozonizing device is positioned, and interfaced with said control unit so as to interrupt the ozone generation following the detection of persons and/or animals.

4. The ozonizing device according to claim 1, wherein said control unit is configured so as to store a group of predefined operating sequences, each sequence providing for the actuation of one of said discharge ozone generator, UV-C lamp and additional ozone generator for a predetermined time period, and comprising a control panel via which the user may select one or more of said predefined operating sequences.

5. The ozonizing device according to claim 3, wherein said control unit is configured so as to store a group of predefined operating sequences, each sequence providing for the actuation of one of said discharge ozone generator, UV-C lamp and additional ozone generator for a predetermined time period, and comprising a control panel via which the user may select one or more of said predefined operating sequences and wherein said presence sensor is interfaced with said control unit so as to be operative or non-operative in accordance with the operating sequence selected by the user.

6. The ozonizing device according to claim 1, wherein said ventilation device comprise at least one fan which is arranged in said first chamber in an intermediate position between the discharge ozone generator and said UV-C lamp.

7. The ozonizing device according to claim 6, wherein said discharge ozone generator comprises an ozone generating plate.

8. The ozonizing device according to claim 7, wherein said ventilation device preferably comprise two fans, said ozone generating plate being arranged in an intermediate position between said two fans.

9. The ozonizing device according to claim 1, wherein there are defined lateral apertures and/or front apertures in the box-shaped body for the intake/discharge of air into/from said first chamber.

10. The ozonizing device according to claim 9, wherein said lateral apertures and/or front apertures are arranged facing said first chamber.

11. The ozonizing device according to claim 4, wherein said control panel which is formed by a touch screen.

12. The ozonizing device according to claim 1, wherein said second chamber is arranged, during use, under said first chamber.

13. The ozonizing device according to claim 1, wherein said discharge ozone generator, ozone generating UV-C lamp and additional ozone generator are mounted on said partition wall.

14. An ozonizing device comprising a box-shaped body which encloses therein a ventilation device, a discharge ozone generator, an ozone-catalysing UV-C lamp for destroying ozone, and an additional ozone generator, the box-shaped body including a partition wall which is intended to define two different chambers in the box-shaped body, a first chamber in which the ventilation device, the discharge ozone generator and the ozone-catalysing UV-C lamp are received and a second chamber, in which the additional ozone generator is received, said additional ozone generator being interfaced with air distribution device which is associated with a diffuser tube for diffusing an air flow which is enriched with ozone outside said ozonizing device by said diffuser tube, said first chamber being configured in such a manner that an air flow through said box-shaped body is defined, the ozonizing device further comprising a control unit which is configured so as to selectively activate, alone or in combination, each of said discharge ozone generator, ozone-catalysing UV-C lamp and additional ozone generator, the ozonizing device further comprising an ionizing device which is interfaced with said control unit, wherein said control unit is configured so as to store a group of predefined operating sequences, each sequence providing for the actuation of one or more of said discharge ozone generator, ozone UV-C lamp, additional ozone generator and ionizing device for a predetermined time period, and comprising a control panel via which the user may select one or more of said predefined operating sequences.

15. The ozonizing device according to claim 14, further comprising a presence sensor which is configured to detect the presence of persons and/or animals inside a room in which the ozonizing device is positioned, and interfaced with said control unit so as to interrupt the ozone generation following the detection of persons and/or animals.

16. The ozonizing device according to claim 15, wherein said presence sensor is interfaced with said control unit so as to be operative or non-operative in accordance with the operating sequence selected by the user.
